(19) United States
(12) Patent Application Publication
(10) Pub. No.: US 2025/0121013 A1
(43) Pub. Date: Apr. 17, 2025

(54) USE OF BACILLUS AMYLOLIQUEFACIENS FOR PREVENTING AND TREATING PARKINSON'S DISEASE

(71) Applicant: Artugen Therapeutics Ltd., Dublin (IE)

(72) Inventors: Susan L. Acton, Lexington, MA (US); Laurent Chesnel, Sant Antonin Noble Val (FR)

(73) Assignee: Artugen Therapeutics Ltd., Dublin (IE)

(21) Appl. No.: 18/688,030

(22) PCT Filed: Sep. 6, 2022
(86) PCT No.: PCT/IB2022/058381

§ 371 Date: Feb. 29, 2024

Related U.S. Application Data
(60) Provisional application No. 63/241,442, filed on Sep. 7, 2021.

(51) Int. Cl. A61K 35/742; A23L 11/50; A23L 33/135; A61K 9/19; A61K 45/06; A61P 25/16

(52) U.S. Cl. CPC A61K 35/742; A23L 11/50; A23L 33/135; A61K 9/19; A61K 45/06; A61P 25/16

(57) ABSTRACT

Disclosed herein are compositions and methods for preventing, ameliorating, or treating Parkinson's disease and/or reducing the severity of one or more risk factors, signs, or symptoms associated with Parkinson's disease. In particular, the technology of the present disclosure relates to methods for administering an effective amount of a composition comprising one or more strains of an operational group Bacillus amyloliquefaciens bacteria, identified as ART24 and ART12, to a subject suffering from or at risk for Parkinson's disease.

### CROSS-REFERENCE TO RELATED APPLICATION

The present application claims priority to U.S. Provisional Patent Application No. 63/241,442, filed on Sep. 7, 2021, the contents of which are hereby incorporated by reference in their entirety.

### TECHNICAL FIELD

The present technology relates to methods and compositions for preventing, ameliorating, or treating Parkinson's disease and/or reducing the severity of one or more risk factors, signs, or symptoms associated with Parkinson's disease. In particular, the present technology relates to administering an effective amount of a composition comprising one or more strains of an operational group Bacillus amyloliquefaciens bacteria, identified as ART24 and ART12, to a subject suffering from or at risk for Parkinson's disease.

### BACKGROUND

The following description is provided to assist the understanding of the reader. None of the information provided or references cited is admitted to be prior art to the compositions and methods disclosed herein.

Parkinson's disease is the second-most common neurodegenerative disease after Alzheimer's disease. Parkinson's disease is a progressive disorder of the neurosystem, primarily impacting movement, but patients may experience non-motor symptoms including, but not limited to, cognitive decline, speech issues, sleep issues, and mood issues. Parkinson's disease is primarily characterized by the death or impairment of dopamine-producing neurons in the midbrain that control movement, although there is also a general loss of norepinephrine production. There is no known cure for Parkinson's disease. Accordingly, there is a need for additional therapies for preventing, treating, or reducing the risk of developing Parkinson's disease.

### SUMMARY

In one aspect, the disclosure of the present technology provides a method for treating or preventing Parkinson's disease (PD) in a subject in need thereof, the method comprising administering to the subject a therapeutically effective amount of a pharmaceutical composition comprising bacterial strain ART24 (NCIMB Accession No. 43088), bacterial strain ART12 (NCIMB Accession No. 43087), or a combination thereof.

In some embodiments, the bacterial strain is lyophilized. In some embodiments, the bacterial strain is in the form of a spore. In some embodiments, the bacterial strain is in vegetative form. In some embodiments, the bacterial strain is a mixture of the strain in the form of a spore and in the vegetative form.

In some embodiments, the bacterial strain is formulated as a dietary supplement. In some embodiments, the bacterial strain is formulated as an edible product. In some embodiments, the edible product further comprises a carrier, vehicle, or excipient. In some embodiments, the edible product further comprises a fermented food product, soybean, mushroom, mung bean, locus bean, rice, or extracts thereof. In some embodiments, the soybean is fermented soybean or fermented soybean paste. In some embodiments, the fermented soybean or fermented soybean paste is Cheonggukjang, Douchi, Hawaijar, Bekang, Peruyaan, Tungrymbai, Eoyukjang, Kinema, Aakhone, Miso, Natto, or Thua-nao.

In some embodiments, the PD comprises one or more of tremors, muscle rigidity, slowed movement, poor posture and balance, changes in speech, struggle with fine motor skills, cognitive changes, constipation, early satiety, excessive sweating, fatigue, seborrheic dermatitis, hallucinations and delusions, orthostatic hypotension, loss of sense of smell or taste, depression, anxiety, apathy, irritability, insomnia, excessive daytime sleepiness, REM sleep behavior disorder, vivid dreams, and Restless Legs Syndrome, vision problems, and weight loss.

In some embodiments, treatment or prevention comprises one or more of increasing striatal dopamine levels or increasing striatal dopamine metabolites such as DOPAC or homovanillic acid levels.

In some embodiments, the pharmaceutical composition is administered orally.

In some embodiments, the method further comprises separately, sequentially, or simultaneously administering one or more agents selected from the group consisting of a dopamine decarboxylase inhibitor, a dopamine precursor, a dopamine agonist, a cathechol-o-methyltransferase inhibitor, a monoamino oxidase-B inhibitor, an adenosine 2A antagonist, and anticholinergic, or an N-methyl-D-aspartate antagonist.

In another aspect, the disclosure of the present technology provides a use of a composition in the preparation of a medicament for treating Parkinson's Disease (PD) in a subject in need thereof, wherein the composition comprises bacterial strain ART24 (NCIMB Accession No. 43088), bacterial strain ART12 (NCIMB Accession No. 43087), or a combination thereof.

In some embodiments, the bacterial strain is lyophilized. In some embodiments, the bacterial strain is in the form of a spore. In some embodiments, the bacterial strain is in vegetative form. In some embodiments, the bacterial strain is a mixture of the strain in the form of a spore and in the vegetative form.

In some embodiments, the bacterial strain is formulated as a dietary supplement. In some embodiments, the bacterial strain is formulated as an edible product. In some embodiments, the edible product further comprises a carrier, vehicle, or excipient. In some embodiments, the edible product further comprises a fermented food product, soybean, mushroom, mung bean, locus bean, rice, or extracts thereof. In some embodiments, the soybean is fermented soybean or fermented soybean paste. In some embodiments, the fermented soybean or fermented soybean paste is Cheonggukjang, Douchi, Hawaijar, Bekang, Peruyaan, Tungrymbai, Eoyukjang, Kinema, Aakhone, Miso, Natto, or Thua-nao.

In some embodiments, the PD comprises one or more of tremors, muscle rigidity, slowed movement, poor posture and balance, changes in speech, struggle with fine motor skills, cognitive changes, constipation, early satiety, excessive sweating, fatigue, seborrheic dermatitis, hallucinations and delusions, orthostatic hypotension, loss of sense of smell or taste, depression, anxiety, apathy, irritability, insomnia, excessive daytime sleepiness, REM sleep behavior disorder, vivid dreams, and Restless Legs Syndrome, vision problems, and weight loss.

In some embodiments, treatment or prevention comprises one or more of increasing striatal dopamine levels or increasing striatal dopamine metabolites such as DOPAC or homovanillic acid levels.

In some embodiments, the pharmaceutical composition is formulated for oral administration.

In some embodiments, the composition for use further comprises separately, sequentially, or simultaneously administering one or more agents selected from the group consisting of a dopamine decarboxylase inhibitor, a dopamine precursor, a dopamine agonist, a cathechol-o-methyltransferase inhibitor, a monoamino oxidase-B inhibitor, an adenosine 2A antagonist, and anticholinergic, or an N-methyl-D-aspartate antagonist.

In another aspect, the technology of the present disclosure provides a composition used for treating Parkinson's Disease (PD) in a subject in need thereof, wherein the composition comprises bacterial strain ART24 (NCIMB Accession No. 43088), bacterial strain ART12 (NCIMB Accession No. 43087), or a combination thereof.

In some embodiments, the bacterial strain is lyophilized. In some embodiments, the bacterial strain is in the form of a spore. In some embodiments, the bacterial strain is in vegetative form. In some embodiments, the bacterial strain is a mixture of the strain in the form of a spore and in the vegetative form.

In some embodiments, the bacterial strain is formulated as a dietary supplement. In some embodiments, the bacterial strain is formulated as an edible product. In some embodiments, the edible product further comprises a carrier, vehicle, or excipient. In some embodiments, the edible product further comprises a fermented food product, soybean, mushroom, mung bean, locus bean, rice, or extracts thereof. In some embodiments, the soybean is fermented soybean or fermented soybean paste. In some embodiments, the fermented soybean or fermented soybean paste is Cheonggukjang, Douchi, Hawaijar, Bekang, Peruyaan, Tungrymbai, Eoyukjang, Kinema, Aakhone, Miso, Natto, or Thua-nao.

In some embodiments, the PD comprises one or more of tremors, muscle rigidity, slowed movement, poor posture and balance, changes in speech, struggle with fine motor skills, cognitive changes, constipation, early satiety, excessive sweating, fatigue, seborrheic dermatitis, hallucinations and delusions, orthostatic hypotension, loss of sense of smell or taste, depression, anxiety, apathy, irritability, insomnia, excessive daytime sleepiness, REM sleep behavior disorder, vivid dreams, and Restless Legs Syndrome, vision problems, and weight loss.

In some embodiments, treatment or prevention comprises one or more of increasing striatal dopamine levels or increasing striatal dopamine metabolites such as DOPAC or homovanillic acid levels.

In some embodiments, the pharmaceutical composition is formulated for oral administration.

In some embodiments, the use further comprises separately, sequentially, or simultaneously administering one or more agents selected from the group consisting of a dopamine decarboxylase inhibitor, a dopamine precursor, a dopamine agonist, a cathechol-o-methyltransferase inhibitor, a monoamino oxidase-B inhibitor, an adenosine 2A antagonist, and anticholinergic, or an N-methyl-D-aspartate antagonist.

In some embodiments, the bacterial strain is ART24.

In some embodiments, the bacterial strain is ART12.

### BRIEF DESCRIPTION OF THE DRAWINGS

FIG. 1A is a chart showing the measured content of dopamine (pg/mg of tissue) in the striatum of mice treated with vehicle and MPTP and test articles (ART24). ****P<0.0001, *P<0.05, one-way ANOVA followed by uncorrected Fisher's LSD test, as compared to Saline/MPTP group, n=10/group.

FIG. 1B is a chart showing the measured content of DOPAC (pg/mg of tissue) in the striatum of mice treated with vehicle and MPTP and test articles (ART24). *P<0.05, ****P<0.0001, one-way ANOVA followed by uncorrected Fisher's LSD test, as compared to Saline/MPTP group, n=10/group.

FIG. 2 is a chart showing the effect of simulated gastric fluid (pH 3) on ART24 spores.

FIG. 3A is a chart showing TNF-α release after 24 hours LPS exposure (% of intoxicated) to primary rat embryo mesencephalic cells (microglia, astrocytes, and neurons). * p<0.05 compared to LPS intoxicated group.

FIG. 3B is a chart showing IL-10 release after 24 hours LPS exposure (% of intoxicated) to primary rat embryo mesencephalic cells (microglia, astrocytes, and neurons). * p<0.05 compared to LPS intoxicated group.

FIG. 3C is a chart showing tyrosine hydroxylase (TH) positive neurons after 5 days LPS exposure (% of control) to primary rat embryo mesencephalic cells (microglia, astrocytes, and neurons). * p<0.05 compared to LPS intoxicated group.

FIGS. 4A-4C are charts showing the results of a locomotion activity analysis in zebrafish after treatment with MPTP and ART24. Distance moved (mm) in response to alternating dark and light phases during 50 minutes (10 min/phase) shown as a total (FIG. 4A) and as distance moved per minute (FIG. 4B). FIG. 4C is a chart showing the percentage of larvae responding to each of the three tap stimuli performed. A larva is considered to respond when its movement after the tap is above 1 mm. Error bars show the maximum and minimum values in FIG. 4A and FIG. 4C, SEM in FIG. 4B. Asterisks indicate significance after one-way ANOVA followed by a Dunnet test comparing against MPTP treatment. N=16 except for DMSO and MPTP (alone) that is N=22 and N=26, respectively. “Bacteria”=ART24.

FIG. 5 is a chart showing the striatal homovanillic acid (HVA) content (pg/mg of tissue) in mice treated with vehicle, MPTP, ART12, or ART24. ****P<0.0001, one-way ANOVA followed by uncorrected Fisher's LSD test, as compared to Saline/MPTP group, n=15/group.

FIG. 6A is a schematic image depicting the 6 clusters analyzed in the tuberculum region of untreated and ART24-treated alpha-synuclein transgenic zebrafish larvae. The image on the left is a representative image of a control larvae without TH expression in regions 1 and 2. The image on the right is a representative image of an ART24 treated larvae with TH expression in regions 1 and 2.

FIG. 6B is a scatter plot of the normalized area of TH expression in the tuberculum region of untreated and ART24-treated alpha-synuclein transgenic zebrafish larvae. The area has been normalized against the average of the control group. The asterisk indicates significance after a Student's t-test. *p<0.05. Error bars show the maximum and minimum values.

FIG. 6C is a graph depicting the percentage of alpha-synuclein transgenic zebrafish larvae with expression of tyrosine hydroxylase in dopamine neurons of the regions 1 and 2 of the tuberculum area. The asterisk indicates significance after a Fisher's exact test. *p<0.05. N=82 and 73 for control and treated groups, respectively.

FIG. 7A is a schematic of the behavioral protocol for zebrafish motor movement analysis. Shaded arrows represent dark phases whereas non-shaded arrows represent light phases.

FIGS. 7B and 7C are charts showing the distance moved (mm) by zebrafish in the presence and absence of MPTP and ART12 or ART24 in response to alternating dark and light phases during 50 minutes (10 min/phase) shown as a total (FIG. 7B) and as distance moved per minute (FIG. 7C).

FIG. 7D is a chart showing the percentage of zebrafish larvae responding to each of the three tap stimuli performed. For FIGS. 7B and 7D, error bars show the maximum and minimum values, SEM in FIG. 7C. Asterisks in FIG. 7B indicate significance after one-way ANOVA followed by Tukey's multiple comparison test. *p<0.05; **p<0.01; ***p<0.001. N=24 in DMSO and MPTP-only groups; N=72 in ART12 and ART24 groups.

FIG. 8A is a graphical representation of Parkinson's disease gene expression in mouse MPTP-treated striatum with or without ART12 treatment. Each column represents a mouse striatum (gray label (the first 15 columns from left to right): ART12 treated; yellow label (the next 15 columns from left to right): control). Each row represents a Parkinson's related gene (Kyoto Encyclopedia of Genes and Genome [KEGG] group). Data are normalized and red indicates higher than average expression while blue indicates lower than average expression. Gene Set Enrichment Analysis (GSEA) identified Parkinson's disease genes as significantly different in the striatum between the ART12-treated and control MPTP mice.

FIG. 8B is a graphical representation of Parkinson's disease gene expression in mouse MPTP-treated colon with ART12 treatment (gray columns (first 15 columns from left to right)) or without treatment (yellow columns (next 15 columns from left to right). Each row represents a Parkinson's related gene (Kyoto Encyclopedia of Genes and Genome [KEGG] group). Data are normalized and red indicates higher than average expression while blue indicates lower than average expression. Gene Set Enrichment Analysis (GSEA) identified Parkinson's disease genes as significantly different in the colon between the ART12-treated and control MPTP mice.

FIG. 8C is a graphical representation of Parkinson's disease gene expression in mouse MPTP-treated colon with ART24 treatment (gray columns (first 15 columns from left to right)) or without treatment (yellow columns (next 15 columns from left to right)). Each row represents a Parkinson's related gene (Kyoto Encyclopedia of Genes and Genome [KEGG] group). Data are normalized and red indicates higher than average expression while blue indicates lower than average expression. Gene Set Enrichment Analysis (GSEA) identified Parkinson's disease genes as significantly different in the colon between the ART24-treated and control MPTP mice.

### DETAILED DESCRIPTION

It is to be appreciated that certain aspects, modes, embodiments, variations and features of the present technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

### I. Definitions

The following terms are used herein, the definitions of which are provided for guidance.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, “administration” of an agent, drug, bacterial strain or spore thereof, or composition of the present technology to a subject includes any route of introducing or delivering to a subject a compound to perform its intended function. Administration can be carried out by any suitable route, including orally, intranasally, parenterally (intravenously, intramuscularly, intraperitoneally, or subcutaneously), topically, or by inhalation. In some embodiments, the compositions of the present technology are formulated for enteric administration. In some embodiments, the compositions are formulated for oral, sublingual, or rectal delivery. In some embodiments, the compositions are formulated for use as a probiotic. In some embodiments, the compositions are formulated for use as a live biotherapeutic. As used herein, administration includes self-administration and administration by another.

As used herein, “ART24” refers to a bacterial strain, or spore thereof, having been deposited under NCIMB Accession No. 43088, or compositions comprising the strain. ART24 is considered a member of an “operational group B. amyloliquefaciens” that comprises the soil-borne B. amyloliquefaciens, and plant associated Bacillus siamensis and Bacillus velezensis.

As used herein, “ART12” refers to a bacterial strain, or spore thereof, having been deposited under NCIMB Accession No. 43087, or compositions comprising the strain. ART12 is considered a member of an “operational group B. amyloliquefaciens” that comprises the soil-borne B. amyloliquefaciens, and plant associated Bacillus siamensis and Bacillus velezensis.

As used herein, the terms “effective amount,” or “therapeutically effective amount,” and “pharmaceutically effective amount” refer to a quantity sufficient to achieve a desired therapeutic and/or prophylactic effect, e.g., an amount which results in the prevention of a disease, condition, and/or symptom(s) thereof. In the context of therapeutic or prophylactic applications, the amount of a composition administered to the subject will depend on the type and severity of the disease and on the characteristics of the subject, such as general health, age, sex, body weight, and tolerance to the composition drugs. It will also depend on the degree, severity, and type of disease or condition. The skilled artisan will be able to determine appropriate dosages depending on these and other factors. In some embodiments, multiple doses are administered. Additionally or alternatively, in some embodiments, multiple therapeutic compositions or compounds (e.g., pharmaceutical compositions comprising the bacterial strain alone or in combination with additional active agents, such as carbidopa-levodopa, amantadine, pramipexole, ropinirole, rotigotine, apomorphine, selegiline, rasagiline, safinamide, entacapone, opicapone, benztropine, trihexyphenidyl) are administered. In the methods described herein, compositions comprising the bacterial strain(s) of the present technology, or spores thereof, may be administered to a subject having one or more signs, symptoms, or risk factors of Parkinson's disease, including, but not limited to, tremors, muscle rigidity, slowed movement, poor posture and balance, changes in speech, struggle with fine motor skills, cognitive changes (e.g., difficulty with attention, planning, memory, dementia), digestive problems (such as constipation), early satiety, excessive sweating, fatigue, seborrheic dermatitis, hallucinations and delusions, orthostatic hypotension, loss of sense of smell or taste, mood disorders (such as depression, anxiety, apathy, and irritability), sleep disorders (such as insomnia, excessive daytime sleepiness, REM sleep behavior disorder, vivid dreams, and Restless Legs Syndrome), vision problems (especially when attempting to read items up close), and weight loss. For example, a “therapeutically effective amount” of the compositions of the present technology, includes levels at which the presence, frequency, or severity of one or more signs, symptoms, or risk factors of Parkinson's disease are, at a minimum, ameliorated. In some embodiments, a therapeutically effective amount reduces or ameliorates the physiological effects of Parkinson's disease, and/or the risk factors of Parkinson's disease, and/or the likelihood of developing Parkinson's disease. In some embodiments, a therapeutically effective amount is achieved by multiple administrations. In some embodiments, a therapeutically effective amount is achieved with a single administration.

As used herein, the terms “freeze-dried” or “freeze-drying” and “lyophilized” or “lyophilization” are used interchangeably and refer to a process that removes water from a product after it is frozen and placed under a vacuum and the products produced therefrom.

As used herein, “pharmaceutically acceptable carrier and/or diluent” or “pharmaceutically acceptable excipient” includes but is not limited to solvents, dispersion media, coatings, antibacterial and antifungal agents, isotonic and absorption delaying agents, and the like. In some embodiments, the pharmaceutically acceptable carrier comprises a polysaccharide, locust bean gum, an anionic polysaccharide, a starch, a protein, sodium ascorbate, glutathione, trehalose, sucrose, or pectin. In some embodiments, the polysaccharide comprises a plant, animal, algal, or microbial polysaccharide. In some embodiments, the polysaccharide comprises guar gum, inulin, amylose, chitosan, chondroitin sulphate, an alginate, or dextran. In some embodiments, the starch comprises rice starch. The use of such media and agents for biologically active substances is well known in the art. Further details of excipients are provided below. Supplementary active ingredients, such as antimicrobials, for example antifungal agents, can also be incorporated into the compositions.

As used herein, “pharmaceutically acceptable excipient” refers to substances and compositions that do not produce an adverse, allergic, or other untoward reaction when administered to an animal or a human. As used herein, the term includes all inert, non-toxic, liquid or solid fillers, or diluents that do not react with the therapeutic substance of the invention in an inappropriate negative manner, solvents, dispersion media, coatings, antibacterial and antifungal agents, isotonic and absorption delaying agents, preservatives and the like, for example liquid pharmaceutical carriers e.g., sterile water, saline, sugar solutions, Tris buffer, ethanol and/or certain oils.

As used herein, “probiotic” refers to bacteria comprising a component of the transient or endogenous flora of a subject administered to confer a beneficial prophylactic and/or therapeutic effect on the subject. Probiotics are generally known to be safe by those skilled in the art. In some embodiments, “probiotics” include “live biotherapeutic products.”

As used herein, “prevention,” “prevent,” or “preventing” of a disorder or condition refers to, in a statistical sample, reduction in the occurrence or recurrence of the disorder or condition in treated subjects/samples relative to an untreated controls, or refers delays the onset of one or more symptoms of the disorder or condition relative to the untreated controls.

As used herein “subject” and “patient” are used interchangeably. In some embodiments, the subject is an animal subject. In some embodiments, the animal subject is a mammal. In some embodiments, the mammalian subject is a human.

As used herein, the term “simultaneous” administration refers to the administration of at least two agents by the same route and at the same time or at substantially the same time.

As used herein, the term “separate” administration refers to an administration of at least two agents at the same time or at substantially the same time by different routes.

As used herein, the term “sequential” administration refers to administration of at least two agents at different times, the administration route being identical or different. More particularly, sequential use refers to the whole administration of one agent before administration of the other agent(s) commences. It is thus possible to administer one of the agents over several minutes, hours, or days before administering another.

A “synergistic therapeutic effect” refers to a greater-than-additive therapeutic effect which is produced by a combination of at least two therapeutic agents, and which exceeds that which would otherwise result from the individual administration of the agents. For example, use of the bacterial strain of the present technology in conjunction with other agents for the treatment of Parkinson's disease may result in a greater than additive therapeutic effect. In some embodiments, the synergistic effect may permit the use of lower doses of the bacterial strain of the present technology and/or other agents than would be required if each were used alone.

“Treating,” “treat,” “treated,” or “treatment” of a disease or disorder includes: (i) inhibiting the disease or disorder, i.e., arresting its development; (ii) relieving the disease or disorder, i.e., causing its regression; (iii) slowing progression of the disorder; and/or (iv) inhibiting, relieving, or slowing progression of one or more symptoms of the disease or disorder.

It is to be appreciated that the various modes of treatment or prevention of medical diseases and conditions as described are intended to mean “substantial,” which includes total but also less than total treatment or prevention, and wherein some biologically or medically relevant result is achieved.

### II. Parkinson's Disease

Parkinson's disease is a progressive disorder of the neurosystem, primarily impacting movement. In addition to motor symptoms, such as slowness of movement, tremor, stiffness and postural instability, patients may develop other health problems related to Parkinson's. These symptoms are diverse and are collectively referred to as non-motor symptoms. Parkinson's disease is primarily characterized by the death or impairment of dopamine-producing neurons in the midbrain that control movement, although there is also a general loss of norepinephrine production.

Early symptoms include minor tremors, frequently in a limb and often in the hand and fingers. Symptoms generally being on one side of the body before spreading throughout the individual. Over time, symptoms can worsen and often include muscle rigidity, slowed movement, poor posture and balance, changes in speech, and struggle with fine motor skills. One or more of a diverse set of non-motor symptoms may develop in Parkinson's disease patients. These non-motor symptoms include, but are not limited to, cognitive changes (e.g., difficulty with attention, planning, memory, dementia), digestive problems (such as constipation) early satiety, excessive sweating, fatigue, seborrheic dermatitis, hallucinations and delusions, orthostatic hypotension, loss of sense of smell or taste, mood disorders (such as depression, anxiety, apathy, and irritability), sleep disorders (such as insomnia, excessive daytime sleepiness, REM sleep behavior disorder, vivid dreams, and Restless Legs Syndrome), vision problems (especially when attempting to read items up close), and weight loss. There is no specific test for diagnosis of Parkinson's disease, and diagnosis is typically carried out via a neurological and physical exam for the disease symptoms. Tests for other conditions, such as MRIs and brain ultrasounds, may be used to rule out other conditions that present with similar symptoms. Diagnosis in mild or early cases may require multiple follow-up appointments with a neurologist who can evaluate changes in symptoms over time. Alternatively, temporary treatment with a Parkinson's medication, such as a carbidopa-levodopa, can be used as a diagnostic tool, with significant improvement in symptoms confirming a diagnosis of Parkinson's disease.

Parkinson's disease cannot be cured with current therapeutics, but medications can help control symptoms. Frequently treatments act as dopamine substitute or increase dopamine production in the brain. One of the most effective treatments, carbidopa-levodopa, is a pro-drug that crosses the blood brain barrier before it is converted into dopamine. Amantadine can be used to treat early mild symptoms of Parkinson's disease and can also be used to counteract side effects from carbidopa-levodopa, such as involuntary movements. Dopamine agonists that mimic the effect of dopamine are also commonly used, including pramipexole, ropinirole, rotigotine, and apomorphine. Another class of treatments include inhibitors that target dopamine-degrading enzymes, such as selegiline, rasagiline, and safinamide, which all inhibit monoamine oxidase B, and entacapone and opicapone, which both inhibit catechol o-methyltransferase. Anticholinergics, such as benztropine and trihexyphenidyl, are infrequently prescribed for tremor control due to side effects but are still available. In cases of advanced Parkinson's disease or for patients with unstable medication responses physicians may use deep brain stimulation surgery with implanted electrodes to treat symptoms via electrical pulses.

In some embodiments, the present technology provides methods and compositions for the treating or preventing Parkinson's disease, including reducing the severity of one or more risk factors, signs, or symptoms associated with Parkinson's disease. In some embodiments the compositions comprise strain ART24 and/or ART12, or spores thereof.

### III. ART24 and ART12

The technology of the present disclosure relates to the use of the ART24 strain and/or ART12 strain, or spores thereof, to treat or prevent Parkinson's disease. ART24 refers to a bacterial strain, or spore thereof, characterized by NCIMB Accession No. 43088, or compositions comprising the strain. ART12 refers to a bacterial strain, or spore thereof, having been deposited under NCIMB Accession No. 43087, or compositions comprising the strain.

In some embodiments, the bacterial strain(s) of the present technology, or spores thereof, is used in methods and compositions for treating or preventing Parkinson's disease. In some embodiments, the bacterial strain(s) comprises a probiotic for preventing or controlling Parkinson's disease. In some embodiments, compositions of the present technology comprise vegetative bacterial cells. In some embodiments, compositions of the present technology comprise bacterial spores. In some embodiments, compositions of the present technology comprise a combination of vegetative bacterial cells and bacterial spores. In some embodiments, the compositions of the present technology comprise an ART24 and/or ART12 bacterial strain that has been physically destructed or lysed. In some embodiments, the compositions of the present technology comprise an isolated fraction of lysed ART24 and/or ART12 bacteria.

In some embodiments, the bacterial strain(s) of the present technology, or spores thereof, is used in methods and edible product compositions for treating or preventing Parkinson's disease in a subject in need thereof. In some embodiments, the bacterial strain comprises a dietary supplement for preventing or controlling Parkinson's disease in a subject in need thereof.

### IV. Therapeutic and Prophylactic Methods

The following discussion is presented by way of example only, and is not intended to be limiting.

One aspect of the present technology includes methods of treating or preventing Parkinson's disease in a subject diagnosed as having, suspected as having, or at risk of having Parkinson's disease. In therapeutic applications, compositions or medicaments comprising the ART24 and/or ART12 bacterial strain, or spores thereof, are administered to a subject suspected of, or already suffering from such a disease (such as, e.g., subjects exhibiting one or more signs or symptoms of Parkinson's disease), in an amount sufficient to cure, or at least partially arrest, the signs or symptoms of the disease, including its complications and intermediate pathological phenotypes in development of the disease.

Subjects suffering from Parkinson's disease can be identified by any or a combination of diagnostic or prognostic assays known in the art. For example, typical symptoms of Parkinson's disease include, but are not limited to, tremors, muscle rigidity, slowed movement, poor posture and balance, changes in speech, struggle with fine motor skills, cognitive changes (e.g., difficulty with attention, planning, memory, dementia), digestive problems (such as constipation), early satiety, excessive sweating, fatigue, seborrheic dermatitis, hallucinations and delusions, orthostatic hypotension, loss of sense of smell or taste, mood disorders (such as depression, anxiety, apathy, and irritability), sleep disorders (such as insomnia, excessive daytime sleepiness, REM sleep behavior disorder, vivid dreams, and Restless Legs Syndrome), vision problems (especially when attempting to read items up close), and weight loss.

In some embodiments, Parkinson's disease subjects treated with the bacterial strain(s) of the present technology, or spores thereof, will show amelioration or elimination of one or more of the following symptoms: tremors, muscle rigidity, slowed movement, poor posture and balance, changes in speech, struggle with fine motor skills, cognitive changes (e.g., difficulty with attention, planning, memory, dementia), digestive problems (such as constipation), early satiety, excessive sweating, fatigue, seborrheic dermatitis, hallucinations and delusions, orthostatic hypotension, loss of sense of smell or taste, mood disorders (such as depression, anxiety, apathy, and irritability), sleep disorders (such as insomnia, excessive daytime sleepiness, REM sleep behavior disorder, vivid dreams, and Restless Legs Syndrome), vision problems (especially when attempting to read items up close), and weight loss.

In one aspect, the present technology provides a method for preventing or delaying the onset of Parkinson's disease or symptoms of Parkinson's disease in a subject at risk of having Parkinson's disease. In some embodiments, the bacterial strain(s) of the present technology is formulated as a probiotic useful as a food supplement and for re-establishing beneficial bacteria in the intestinal tract. In some embodiments, the bacterial strain(s) of the present technology is formulated as a live biotherapeutic product useful in pharmaceutical applications. In some embodiments, the bacterial strain(s) of the present technology is formulated as a live biotherapeutic edible product useful in pharmaceutical applications.

### V. Modes of Administration and Effective Dosages

Compositions of the present technology for use in preventing, ameliorating, or treating Parkinson's disease and/or reducing the severity of one or more risk factors, signs, or symptoms associated with Parkinson's disease include live probiotic ART24 and/or ART12 bacteria according to the present technology, provided in the form of vegetative cells and/or spores. In some embodiments, the bacterial strain(s) is lyophilized. The compositions of the present technology are administered to the subject in effective amounts (i.e., amounts that have desired therapeutic effect). The dose and dosage regimen will depend upon the degree of disease, the characteristics of the ART24 and/or ART12 strain used, e.g., its therapeutic index, the subject, and the subject's history. The effective amount may be determined during pre-clinical trials and clinical trials by methods familiar to physicians and clinicians.

Compositions of the present technology may be formulated for adding to food, or used directly as a food supplement. The formulation may further include other probiotic agents or nutrients for promoting spore germination and/or bacterial growth.

Edible products of the present technology may be formulated for adding to food, or used directly as a dietary supplement. In some embodiments, the edible product comprises a fermented food product, soybean, mushroom, mung bean, locus bean, or rice. In some embodiments, the soybean is fermented soybean or fermented soybean paste. The formulation may further include other probiotic agents or nutrients for promoting spore germination and/or bacterial growth.

Additional components of the compositions of the present technology may include a preservative selected from the group consisting of sucrose, sodium ascorbate, and glutathione. In some embodiments the preservative is a cryoprotectant selected from the group consisting of a nucleotide, a disaccharide, a polyol, and a polysaccharide. In some embodiments, the cryoprotectant is selected from the group consisting of inosine-5′-monophosphate (IMP), guanosine-5′-monophosphate (GMP), adenosine-5′-monophosphate (AMP), uranosine-5′-monophosphate (UMP), cytidine-5′-monophosphate (CMP), adenine, guanine, uracil, cytosine, guanosine, uridine, cytidine, hypoxanthine, xanthine, orotidine, thymidine, inosine, trehalose, maltose, lactose, sucrose, sorbitol, mannitol, dextrin, inulin, sodium ascorbate, glutathione, skim milk, and cryoprotectant 18.

The ART24 and/or ART12 bacterial strain described herein can be incorporated into pharmaceutical compositions for administration, singly or in combination, and given to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions. Carriers can be solid-based dry materials for formulations in powdered form, and can be liquid or gel-based materials for formulations in liquid or gel forms, which forms depend, in part, upon the routes or modes of administration. In some embodiments, the pharmaceutically acceptable carrier comprises a polysaccharide, locust bean gum, an anionic polysaccharide, a starch, a protein, sodium ascorbate, glutathione, trehalose, sucrose, or pectin. In some embodiments, the polysaccharide comprises a plant, animal, algal, or microbial polysaccharide. In some embodiments, the polysaccharide comprises guar gum, inulin, amylose, chitosan, chondroitin sulphate, an alginate, or dextran. In some embodiments, the starch comprises rice starch.

Pharmaceutical compositions are typically formulated to be compatible with its intended route of administration. Examples of routes of administration include enteric (e.g., oral, sublingual, rectal) administration. A therapeutic composition can be formulated to be suitable for oral administration in a variety of ways, for example in a liquid, a powdered food supplement, a solid food, a packaged food, a wafer, tablets, troches, or capsules, e.g., gelatin capsules, and the like. In some embodiments, the therapeutic compositions of the present technology comprise lyophilized ART24 and/or ART12. In some embodiments, the lyophilized ART24 and/or ART12 is encapsulated. A therapeutic composition can be formulated to be suitable for rectal administration in a variety of ways, for example in a suppository, liquid enema, or foam. Other formulations will be readily apparent to one skilled in the art. pH can be adjusted with acids or bases, such as hydrochloric acid or sodium hydroxide.

Additionally or alternatively, for edible products, pharmaceutical compositions of the present technology may be formulated as dietary supplements, nutraceutical compositions, food additives, food compositions, food compositions in bulk, food additives in bulk, medical foods, or foods for special dietary use, for enteric (e.g., oral, sublingual) administration.

Dosage, toxicity and therapeutic efficacy of any therapeutic agent can be determined by standard pharmaceutical procedures in cell cultures or experimental animals. The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately.

In some embodiments, the “edible products” such as whole soybeans and/or soybean pastes, are inoculated with a concentration of ART24 and/or ART12 ranging from at least about 1×10² colony forming units (CFU)/mL to at least about 1×10¹⁰ CFU/mL, or any value in between. For example, in some embodiments, the edible products are inoculated with at least about 1×10³ CFU/mL to at least about 1×10¹⁰ CFU/mL ART24 and/or ART12, at least about 1×10⁴ CFU/mL to at least about 1×10¹⁰ CFU/mL ART24 and/or ART12, at least about 1×10⁵ CFU/mL to at least about 1×10¹⁰ CFU/mL ART24 and/or ART12, at least about 1×10⁶ CFU/mL to at least about 1×10¹⁰ CFU/mL ART24 and/or ART12, at least about 1×10⁷ CFU/mL to at least about 1×10¹⁰ CFU/mL ART24 and/or ART12, at least about 1×10⁸ CFU/mL to at least about 1×10¹⁰ CFU/mL ART24 and/or ART12, at least about 1×10⁹ CFU/mL to at least about 1×10¹⁰ CFU/mL ART24 and/or ART12. In some embodiments, the edible products are inoculated with at least about 6×10⁸ CFU/mL ART24 and/or ART12. In some embodiments, the edible products are inoculated with at least about 6×10⁹ CFU/mL ART24 and/or ART12. In some embodiments, the edible products are inoculated with at least about 1×10⁴ CFU/mL ART24 and/or ART12. In some embodiments, the edible products are inoculated with at least about 1×10⁴ CFU/mL ART24 and/or ART12. In some embodiments, the edible products are inoculated with at least about 1×10⁶ CFU/mL ART24 and/or ART12. In some embodiments, the edible products are inoculated with at least about 1×10⁸ CFU/mL ART24 and/or ART12. In some embodiments, the edible products are inoculated with at least about 1×10⁹ CFU/mL ART24 and/or ART12.

In some embodiments, the compositions of the present technology contain between 10⁴ to 10¹² colony forming units (CFU) of ART24 and/or ART12 bacterium (i.e., vegetative cell) or bacterial spore. In some embodiments, the methods of the present technology involve the administration of about 10⁴ to about 10¹² viable bacteria or spores per day. In some embodiments, the compositions of the present technology are delivered as lyophilized material or powder to be re-suspended for oral delivery or packaged into capsules. The lyophilized material and capsules may be coated for better enteric stability.

An exemplary treatment regimen entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the subject can be administered a prophylactic regime. In some embodiments, compositions of the present technology are administered to a subject once, twice, or three times per day or more for a certain period of time or until the subject is deemed cured of primary disease, not to be at risk for recurrence of primary disease, or not to be at risk for the disease. In some embodiments, the compositions of the present technology may be administered to the subject for the remainder of the subject's life. In some embodiments, administration is paired with a shortened exposure to agents known in the art for the treatment of Parkinson's disease, such as carbidopa-levodopa, amantadine, pramipexole, ropinirole, rotigotine, apomorphine, selegiline, rasagiline, safinamide, entacapone, opicapone, benztropine, trihexyphenidyl, followed by once, twice, or three times daily dosing or more for a certain period of time or until the patient is deemed cured of primary disease or not to be at risk for recurrence of the disease. In some embodiments, methods of prophylaxis comprise administration of compositions of the present technology once, twice, or three times daily or more for a certain period of time or until the patient is deemed not to be at risk of developing the disease in the case of patients known to be at risk for Parkinson's disease.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

### VI. Combination Therapy with ART24 and/or ART12

In some embodiments, the ART24 and/or ART12 strain of the present technology, or spores thereof, may be combined with one or more additional therapies for the prevention or treatment of Parkinson's disease. Additional therapeutic agents include, but are not limited to, one or more additional therapeutic agents selected from the group consisting of: carbidopa-levodopa, amantadine, pramipexole, ropinirole, rotigotine, apomorphine, selegiline, rasagiline, safinamide, entacapone, opicapone, benztropine, trihexyphenidyl.

In some embodiments, an additional therapeutic agent is administered to a subject in combination with the ART24 and/or ART12 strain of the present technology, or spores thereof, such that a synergistic therapeutic effect is produced. For example, administration of ART24 and/or ART12 with one or more additional therapeutic agents for the prevention or treatment of Parkinson's disease will have greater than additive effects in the prevention or treatment of the disease.

In any case, the multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. In addition, the combination methods, compositions and formulations are not to be limited to the use of only two agents.

### EXAMPLES

The following examples are provided by way of illustration only and not by way of limitation. Those of skill in the art will readily recognize a variety of non-critical parameters that could be changed or modified to yield essentially the same or similar results. The examples should in no way be construed as limiting the scope of the present technology, as defined by the appended claims.

### Example 1: ART24 is Effective in Methods for Treating or Preventing Parkinson's Disease

### Animal Model

This example demonstrates that ART24 is effective in methods for treating or preventing Parkinson's disease in an in vivo mouse model.

### Introduction

MPTP is a potent and selective nigrostriatal dopaminergic neurotoxin that produces many of the neuropathological features of idiopathic Parkinson's disease (PD) in humans, nonhuman primates, and mice. In mice, MPTP produces nigrostriatal dopaminergic degeneration and locomotor impairment. Pharmacological agents that increase dopaminergic function or that block the neurotoxicity of MPTP also attenuate MPTP associated locomotor dysfunction and have been useful in the clinic for treating Parkinson's disease. Moreover, MPTP-mediated toxicity may have a relationship to the mechanisms associated with dopaminergic loss in the disease indicating that this model may also be potentially useful for identifying agents that slow or reduce nigrostriatal dopaminergic loss.

The study was conducted to test the effect of ART24 and trehalose in the MPTP-induced model of PD.

### Experimental Procedures

### Animal Description:

Species: Mouse
Strain: C57Bl/6
Source of Animals: Charles River Labs
Age or weight: 8 weeks
Sex: Male
Randomization: Animals were assigned randomly to treatment groups;
Blinding of Study: The group information was blinded to the scientists.

### Housing and Feeding:

Acclimation/Conditioning:
   Seven days in the Melior animal housing facility prior to the study
Housing:
   Mice were housed on a 12 hr light/dark cycle (lights on 7:00 AM)
   No more than 4 mice per cage
   Ventilated cage rack system
Diet:
   Standard rodent chow and water ad libitum

### Design:

Route(s) of administration: IP for MPTP (1-Methyl-4-phenyl-1,2,3,6-tetrahydropyridine hydrochloride) and vehicle, PO for ART24 and vehicle (trehalose)
Dose Volume(s): 10 mL/kg for MPTP, 0.1 mL for ART24
Formulation(s): PBS for MPTP; saline-based formulation for ART24
Dose Levels: 20 mg/kg for MPTP, for ART24
Dose Frequency: 4 doses (QD) for MPTP; 11 days BID for ART24
Study duration: 11 days
Number of Groups: 4
Number of animals per group: 13
Total number of animals: 52 (57 animals were enrolled in the study)

TABLE 1
Study Design.

| | | | | Group | |
|---|---|---|---|---|---|
| Group | Treatment | MPTP | Treatment | Size | Evaluations/Endpoints |
| 1 | Sham | PBS, 4 doses IP | Saline, | 13 | 3× per week: Body weight and |
| | | QD, for 4 days | PO BID | | clinical observations |
| | | beginning day 0 | from | | 7 days post-MPTP: |
| 2 | MPTP | 4 doses, 20 mg/kg | Day (−3) | 13* | N = 10/group |
| | | IP QD for 4 days | to | | Blood/plasma collection |
| | | beginning day 0 | Day 7 | | Dissection of striata (bilateral), |
| 3 | | | Trebalose, | | Determination of DA/DOPAC |
| | | | BID QD | | in the striata |
| | | | from | | N = 3/group |
| | | | Day (−3) | | Perfusion of animals with saline |
| | | | to Day 7 | | following by PFA fixation |
| 4 | | | ART24, | | Brain extraction, postfix and |
| | | | BID QD | | cryoprotection for IHC (TBD), |
| | | | from | | |
| | | | Day (−3) | | |
| | | | to Day 7 | | |

*Total number of animals dosed in each group was 15 to account for possible loss of animals.

### Methods

MPTP model. MPTP was formulated in phosphate buffered saline (PBS) and administered to mice QD for 4 days at a dose of 20 mg/kg (2 mg/mL delivered as 10 mL/kg).

Tissue dissection for IHC. At termination of the study, randomly selected animals (n=3/group) were deeply anesthetized with isoflurane.
Brain collection:
   Animals were perfused through the heart with ice-cold PBS with Heparin (30 u/ml), pH 7.4. 50 ml of PBS was perfused for each animal.
   50 ml of 20% PFA was perfused;
   Brains were carefully extracted and transferred into 20% PFA solution, postfixed for 48 hrs
   After 48 Hrs, brains were transferred to PBS and treated as recommended by a IHC lab.

Neurotransmitter measurements (DA and DOPAC). 10 mice from each were anesthetized and blood collected into K2EDTA tubes by a cardiac puncture. Brains were extracted and dissected. The rostral part was dissected and striata removed. The striata were frozen and subsequently evaluated for levels of dopamine and metabolites.

Striata were homogenized in 0.3M perchloric acid. The resulting supernatant was analyzed for levels of monoamines by reverse phase HPLC-ECD.

Blood was processed for plasma. Resulting plasma was snap frozen for further analyses.

Data analysis. Data were analyzed using a two-way repeated measures ANOVA with a post hoc Fisher's LSD multiple comparison test. The raw data were also analyzed for outliers using ROUT method (Q=1%). GraphPad Prism 9 was used for data analysis and presentation.

### Results

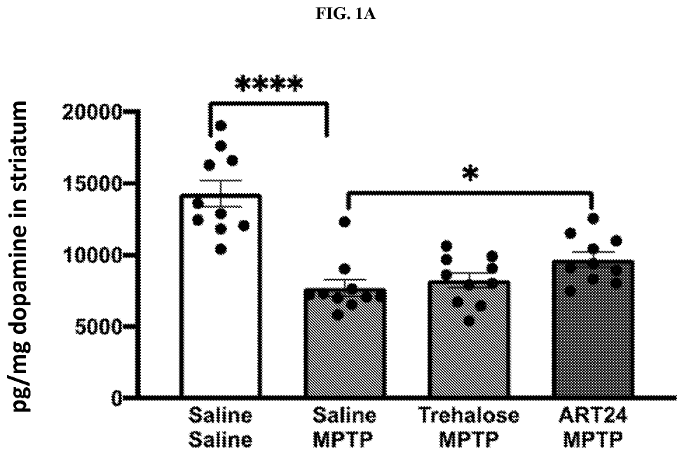

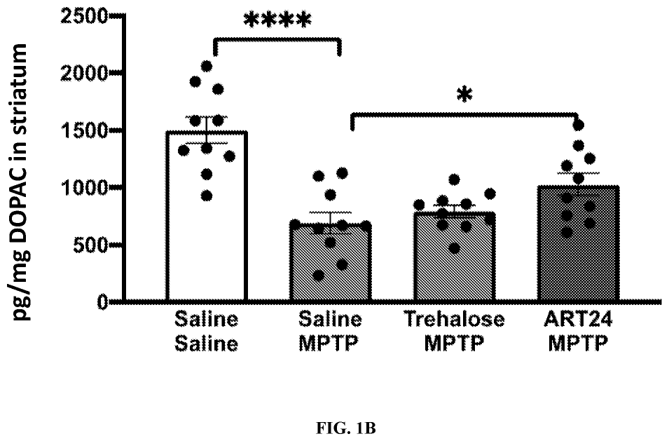

As shown in FIGS. 1A and 1B, treatment with MPTP produced a significant depletion in dopamine and DOPAC content in the striatum compared to saline/vehicle treated animals. Also as shown in FIGS. 1A and 1B, treatment with ART24 resulted in a significant (P<0.05) effect on dopamine (FIG. 1A) and DOPAC (FIG. 1B) concentrations as compared to saline/MPTP dosed animals. Treatment with trehalose did not produce a significant effect on the experimental endpoints (FIGS. 1A and 1B).

Accordingly, these results demonstrate that ART24 is effective in methods for preventing, reducing the risk of, or treating Parkinson's disease in a subject in need thereof.

### Human Subjects

Human subjects diagnosed as having or suspected to have Parkinson's disease and presently displaying one or more symptoms and/or pathologies of Parkinson's disease, are recruited using selection criteria known and accepted in the art.

### Methods of Prevention and Treatment

Subjects are administered ART24 at a dosage and frequency commensurate with the stage and severity of disease. In some embodiments ART24 is administered once daily, once weekly, or once monthly. In some embodiments, ART24 is administered multiple times daily, weekly, or monthly.

To demonstrate methods of prevention and treatment in humans, subjects are administered ART24 prior to or subsequent to the development of symptoms and/or pathologies of Parkinson's' disease and assessed for reversal of symptoms/pathologies or attenuation of expected symptoms/pathologies using methods known in the art.

### Results

It is expected that ART24 will inhibit the worsening of symptoms and/or pathologies of Parkinson's disease in human subjects. These results will show that ART24 is useful and effective for the prevention and treatment of such a disorder.

### Example 2: ART24 is Effective in Methods for Reducing Inflammatory Markers in the Striatum of an Animal Model of Parkinson's Disease

This example prophetically demonstrates that ART24 is effective in methods for reducing inflammatory markers in the striatum of an animal model of Parkinson's disease in an in vivo animal model.

### Methods

Brain Preparation. Mice are exposed to one of three treatment conditions: (1) a PBS and saline negative control; (2) a MPTP and saline positive control for striatum inflammation; and (3) a MPTP and Test Article (“TA”=ART24) condition to assay for the anti-inflammatory impact of ART24 in the striatum. The mice are euthanized, and brains are collected. Mouse brains are sliced into 2 mm portions using a brain slicer, then each portion is embedded into one block to ensure that the region (the striatum) is captured. These sections are dehydrated and infiltrated with paraffin using a tissue processer and immediately embedded in cassettes where 5 m sections are cut using a microtome and mounted on super frost plus slides.

Immunolabeling and Imaging. The 5 m thick brain sections are baked at 60° C. for one hour then rehydrated in a stepwise gradient with ethanol. The sections then undergo IHC to label Ibal+microglia and GFAP by first blocking with 6% donkey serum in 0.2% PBST for one hour, followed by a one hour incubation with goat anti-Ibal (1:250, Abcam) and mouse anti-GFAP (1:200, Thermo Fisher) and one hour of secondary antibody incubation using Donkey anti-goat AF488 and Donkey ant-mouse AF555 (1:500, Thermo Fisher). DAPI (1:4000) is used for the nuclear stain and the slides are coverslipped with 90% glycerol in 1×PBS. Slides are imaged using a Leica Versa 8 slide imager and exposed across two channels (DAPI at 405 nm and Ibal at 488 nm and GFAP at 555 nm) at 40× magnification (0.1625 μm/pixel).

Cell Counting and Quantification. Images of Ibal and GFAP fluorescence from brain slices are background subtracted and segmented to convert into binary images to count cells within the manually drawn striatum. Ibal and GFAP are markers of inflammation known to increase in the MPTP model of PD. For each sample, the density value is averaged across their section count to represent one brain, then that group's difference measured between the other samples. For statistical analysis, one-way ANOVA is performed together with Tukey's multiple comparisons test for post hoc analysis to evaluate significance of difference between groups.

### Results

It is anticipated that these results will show that treatment of mice with ART24 will reduce markers of inflammation, such as Ibal and GFAP, which are known to increase in a model of Parkinson's disease. Accordingly, these results will show that ART24 is effective in methods for preventing, reducing the risk of, or treating Parkinson's disease in a subject in need thereof.

### Example 3: ART24 within EFSA Antibiotic Susceptibility Guidelines and does not Contain Virulence Factors or Pathogenicity Islands

As demonstrated by Table 2, ART24 is within the European Food Safety Authority (EFSA) guidelines. The minimum inhibitory concentration (MIC) of the listed antimicrobials is expressed as μg/mL. Additional studies following EFSA testing recommendations were performed on lyophilized ART24 lots CO-33-10A2 and CO-33-12A2 and Research and Master Cell Banks CO-33-ART24 BHI and CO-33-ART24 SYD, and those results are shown in Table 3.

TABLE 2
ART24 within EFSA antibiotic susceptibility guidelines

| Antimicrobial | ART24 MIC (μg/mL) | EFSA guidelines |
|---|---|---|
| Ampicillin | 0.25 | n.r. |
| Vancomycin | 0.5-1 | 4 |
| Gentamicin* | ≤0.5 | 4 |
| Kanamycin* | ≤2 | 8 |
| Streptomycin | 4 | 8 |
| Erythromycin | 0.12 | 4 |
| Clindamycin | 0.5-1 | 4 |
| Tetracycline | 4 | 8 |
| Chloroamphenicol | 4 | 8 |
| Neomycin* | ≤0.5 | — |
| Penicillin | 0.12-0.5 | — |
| Quinupristin-dalfopristin | 4 | — |
| Linezolid | 0.5 | — |
| Trimethoprim | ≤0.12 | — |
| Ciprofloxacin* | ≤0.25 | — |
| Rifampicin | 0.5 | — |

Average results from N = 6.
*No growth with the lowest concentration in the plate.

TABLE 3
ART24 within EFSA antibiotic susceptibility guidelines

| | MIC in mg/L (interpretation) for | | | |
|---|---|---|---|---|
| | ART24 for all 4 sources^{a} | | | |
| | CO-33 ART | CO-33 ART | CO-33- | CO-33- |
| Antimicrobial | 24 BHI | 024 SYD | 10A2 | 12A2 |
| Ampicillin | ≤0.03 (S) | ≤0.03 (S) | ≤0.03 (S) | ≤0.03 (S) |
| Chloramphenicol | 2 (S) | 2 (S) | 2 (S) | 2 (S) |
| Ciprofloxacin | ≤0.03 (S) | ≤0.03 (S) | ≤0.03 (S) | ≤0.03 (S) |
| Clindamycin | 0.25 (S) | 0.25 (S) | 0.12 (S) | 0.25 (S) |
| Erythromycin | 0.12 (S) | 0.12 (S) | 0.06 (S) | 0.06 (S) |
| Fidaxomicin | 4 | 4 | 2 | 4 |
| Gentamicin | ≤0.06 (S) | ≤0.06 (S) | ≤0.06 (S) | ≤0.06 (S) |
| Kanamycin | ≤0.5 (S) | ≤0.5 (S) | ≤0.5 (S) | ≤0.5 (S) |
| Linezolid | 0.5 | 0.5 | 0.25 | 0.5 |
| Metronidazole | >64 | >64 | >64 | >64 |
| (aerobic) | | | | |
| Metronidazole | 64 | 64 | 64 | 64 |
| (anaerobic) | | | | |
| Neomycin | ≤0.12 | ≤0.12 | ≤0.12 | ≤0.12 |
| Penicillin | ≤0.03 (S) | 0.06 (S) | ≤0.03 (S) | ≤0.03 (S) |
| Quinupristin- | 2 | 2 | 1 | 2 |
| dalfopristin | | | | |
| Rifampicin | 0.25 (S) | 0.25 (S) | 0.25 (S) | 0.25 (S) |
| Streptomycin | 1 (S) | 2 (S) | 1 (S) | 1 (S) |
| Tetracycline | 2 (S) | 2 (S) | 2 (S) | 2 (S) |
| Trimethoprim | ≤0.25 | ≤0.25 | ≤0.25 | ≤0.25 |
| Vancomycin | 0.5 (S) | ≤0.25 (S) | ≤0.25 (S) | ≤0.25 (S) |

^{a}Categorical interpretations using CLSI M45 (2015) breakpoint criteria, as available. MIC interpretations for kanamycin and streptomycin used European Food Safety Authority cutoff values (2012).

Briefly, material from 3-5 colonies of ART24 were selected and suspended in 4 mL of MRD to obtain a cell concentration of ˜1×10⁸ CFU/mL. From this suspension 46 μL was transferred in to 23 mL of 90% Iso-Sensitest Broth (CM0473, Oxoid) containing 10% BHI broth to end with a final inoculum concentration of 5×10⁵ CFU/mL. The wells of the prefabricated 96-well plates were filled with 100 μL of the final suspension. The plates were sealed with an AeraSeal (A9224, Sigma Aldrich) sealing film (to facilitate growth of the aerobic Bacillus. The plates were then incubated for 24 hr at 37° C. The results are recorded after 24 hours as the lowest concentration of the antibiotic to inhibit visible growth. The prefabricated panels VetMIC Lact-1 & Lact-2 (SVA, Uppsala, Sweden) comprising the antibiotics ampicillin, vancomycin, gentamicin, kanamycin, streptomycin, erythromycin, clindamycin, tetracycline, chloramphenicol, neomycin, penicillin, quinupristin-dalfopristin, linezolid, trimethoprim, ciprofloxacin and rifampicin were used.

The absence of antibiotic resistance in the strain of the present technology precludes the risk of horizontal transfer of antibiotic resistance.

Moreover, searches conducted with KEGG, RAST SEED Viewer, IslandViewer 4 and ACT comparisons to known Bacillus species that produce toxins, etc., did not reveal any virulence factors or pathogenicity islands in the genomes of the ART24 strain of the present technology. Accordingly, these results demonstrate the probiotic safety of the strain in that it lacks the propensity to confer resistance to commensals and/or pathogens.

### Example 4: Freeze-Drying (Lyophilization) to Preserve ART24

This example demonstrates that ART24 is useful in compositions and methods wherein the strain is freeze-dried (or lyophilized).

2xSG Sporulation media: Difco nutrient broth 16.0 g/L; KCl, 2.0 g/L; MgSO₄.7H₂O, 0.5 g/L. Adjust the pH to 7.0, autoclave. After autoclaving and cooling add 1 M Ca(NO₃)₂.4H₂O, 1.0 ml/L; 0.1M MnCl₂.4H₂O, 1.0 ml/L; 1 mM FeSO₄.7H₂O, 1.0 ml/L; 50% (w/v) glucose, 2.0 ml/L.

Spore preparation method: Inoculate 50 ml of BHI (1.10493.0500, Merck) medium with cells from a fresh colony of ART24. Grow aerobically in flasks 24 hours at 37° C. with shaking (orbital platform shaker) at 200 rpm. Dilute 1/200 into 1 L 2xSG medium in an appropriate vessel and grow at 37° C. with shaking at 200 rpm. Check samples for spores daily using light microscopy. Phase contrast microscopy is ideal. After 2-3 days >90% of the population should have sporulated. Pellet cells at 9000 rpm for 20 minutes in a centrifuge if possible, keeping temperatures low, otherwise for small quantities a benchtop centrifuge will suffice. Wash and centrifuge the spores with ice-cold water 2-3 times to remove residual nutrients and lyse remaining vegetative cells. Resuspend the spore pellet in 10 mg/ml lysozyme solution and incubate shaking at 37° C. for 1 hour. Wash and centrifuge the spores with ice-cold water 4-6 times to remove residual nutrients and lyse remaining vegetative cells. Freeze dry 3 ml aliquots of spores over 24 hr period in a VirTis Advantage Wizard freeze dryer and store in sealed freeze-drying vials at room temperature for long-term storage.

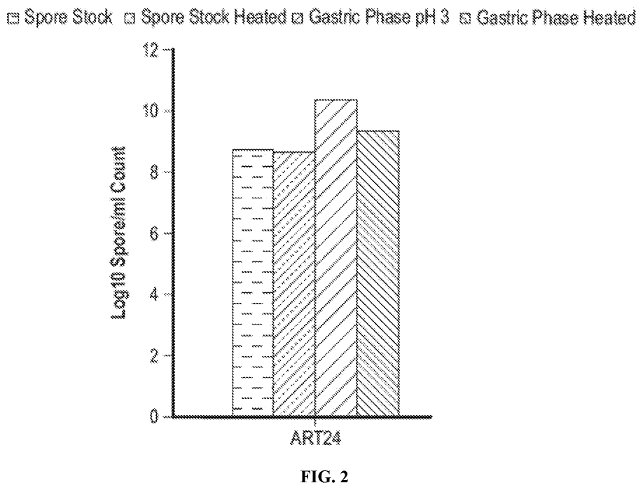

Simulated gastric fluid was prepared based on the SGF outlined by the Infogest COST action containing 37.3 g/L KCl, 68 g/L KH₂PO₄, 84 g/L NaHCO₃, 117 g/L NaCl, 30.5 g/L (NH₄)CO₃, 44.1 g/L CaCl₂) and 2 mL of a 20,000 U/mL solution. As shown in FIG. 2, spores of ART24 survive when exposed to simulated gastric fluid.

As shown in Table 4, spores of ART24 achieve 100% survivability after freeze drying and resuspension.

To test the robustness of the cells, the strain was freeze-dried. Briefly, overnight cultures of ART24 were grown according to methods known in the art and re-suspended in 15% w/v trehalose solution. Cell counts were performed.

The cultures were freeze-dried for 22 hours, after which they were re-suspended in an equivalent volume of water lost. Cell counts were performed. Table 4 shows the survivability results.

TABLE 4
Survivability of ART24 after freeze drying (lyophilization)

| | Pre FD | Post FD |
|---|---|---|
| | (log) | (Log) |
| ART24 | 8.2 | 8.9 |

Accordingly, these results demonstrate that ART24 is useful in compositions and methods wherein the strain is freeze-dried (or lyophilized).

### Example 5: Neuroprotective Effect of ART24 on Damage Induced by Lipopolysaccharide (LPS) on Mesencephalic Culture

This example demonstrates that ART24 has a neuroprotective effect on mesencephalic neurons and is useful in methods for treating or preventing Parkinson's disease.

### Methods

Table 5 outlines the experimental design for this example.

TABLE 5
Experimental design.

| Day | | Tasks |
|---|---|---|
| 0 | | Plating of a primary culture of rat embryo |
| | | mesencephalic cells. This culture contains |
| | | microglia, astrocytes, and neurons. |
| 2 | | Renewal of medium |
| 5 | | Renewal of medium |
| 7 | | Compound application |
| | | (1 h before LPS injury) + LPS exposure |
| 8 | (24 h post-LPS) | Measure TNF-α and IL-1β release |
| 12 | (day 5 post-LPS) | Measure death of dopaminergic neurons |

(Tyrosine hydroxylase positive neurons)

The protocol was performed in 3 independent cultures. For each culture, each condition was performed in sextuplicate for measures at day 12 (day 5 post-LPS) and in triplicate for measures at day 8. Each plate contained 3 types of control to validate the experiment: the control condition treated with vehicle (no injury), the LPS condition treated with vehicle, and the LPS conditions treated with a positive control (e.g., dexamethasone).

Test conditions. ART24 extracts of bacteria resuspended in DMSO:
IPA: Isopropyl alcohol extract of bacterial pellet at the following dilution of the stock: 1:2500×.

### Readouts.

Readout 24 h after LPS injury and treatment with compound: TNF-α, IL-1
Readout after 5 days of LPS injury and treatment with compound: Percentage of tyrosine hydroxylase (TH) positive neurons (dopaminergic neurons).

### Results

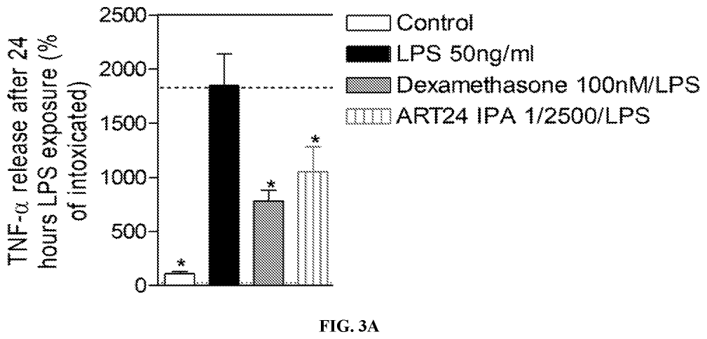

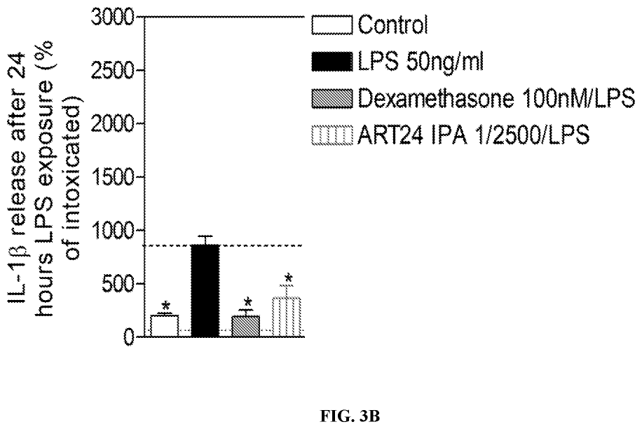

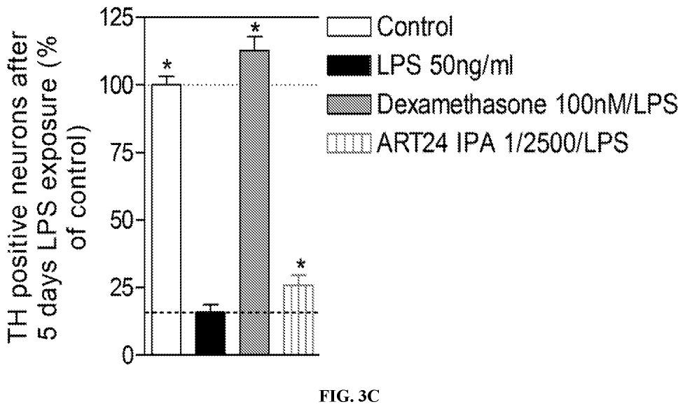

As shown in FIGS. 3A, 3B, and 3C, treatment of mesencephalic neuronal cultures consisting of neurons and glial cells with ART24 reduced TNF-α levels, reduced IL-10 levels, and increased TH positive neurons, respectively, as compared to the LPS alone treated group.

These results demonstrate neuroprotective effects of ART24. Accordingly, these results demonstrate that ART24 is useful in methods for treating or preventing Parkinson's disease.

### Example 6: ART24 is Effective in Methods for Treating or Preventing Parkinson's Disease in an In Vivo Zebrafish Model

This example demonstrates that ART24 is effective in methods for treating or preventing Parkinson's disease (PD) in an in vivo zebrafish model. The study was conducted to test the effect of ART24 in the MPTP-induced model of PD.

### Methods

Zebrafish larvae were dosed with MPTP (250 μM; 96 to 120 hours post fertilization) or sham treated (DMSO only; n=22) in water. ART24 was dosed in water of MPTP-exposed fish (4×10⁶ CFU/mL; n=16). Movement was evaluated with an imaging system during 50 min of testing consisting of 10-min periods of alternating darkness/bright light. Data was analyzed by one-way ANOVA followed by a Dunnet test comparing against MPTP treatment.

### Results

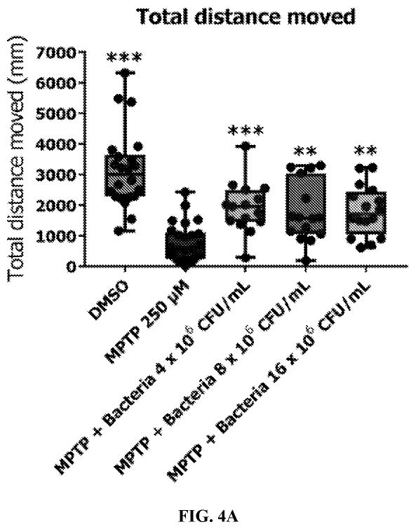

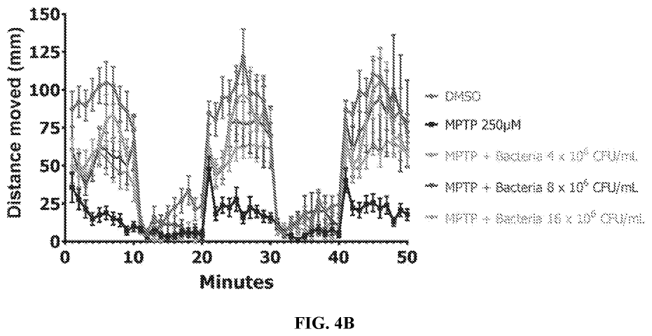

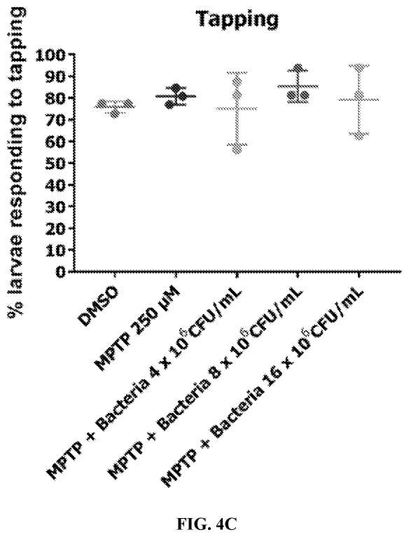

Locomotion activity analysis. As shown in FIGS. 4A and 4B, MPTP-treated Zebrafish larvae had significantly reduced total motor movement distance (24% of sham; p<0.001), which was attenuated by ART24 (61% of sham; p<0.001).

### Conclusion

Treatment with ART24 led to improved motor movement in an MPTP zebrafish model (FIGS. 4A and 4B). Accordingly, these results demonstrate that ART24 is effective in methods for preventing, reducing the risk of, or treating Parkinson's disease in a subject in need thereof.

### Example 7: ART24 and ART12 are Effective in Methods for Treating or Preventing Parkinson's Disease in an In Vivo Mouse Model of MPTP-Induced Parkinson's Disease

This example demonstrates that ART24 and ART12 are effective in methods for treating or preventing Parkinson's disease in an in vivo mouse model.

### Experimental Procedures

### Animal Description:

Species: Mouse
Strain: C57Bl/6
Source of Animals: Charles River Labs
Age or weight: 8 weeks
Sex: Male
Randomization: Animals were assigned randomly to treatment groups;
Blinding of Study: The study was not blinded.

### Housing and Feeding:

Acclimation/Conditioning:
   Not less than five days
Housing:
   Mice were housed on a 12 hr light/dark cycle (lights on 7:00 AM)
   No more than 3 mice per cage
   Ventilated cage rack system
Diet:
   Standard rodent chow and water ad libitum

### Design:

Route(s) of administration: IP for MPTP (1-Methyl-4-phenyl-1,2,3,6-tetrahydropyridine hydrochloride) and vehicle, PO for ART24 and ART12, and vehicle (trehalose)
Dose Volume(s): 10 mL/kg for MPTP, 0.1 mL for ART24 and ART12
Formulation(s): PBS for MPTP; saline-based formulation for ART24 and ART12
Dose Levels: 20 mg/kg for MPTP, for ART24 and ART12 5×10⁸ CFU/dose
Dose Frequency: 4 doses (QD) for MPTP; 10 days BID for ART24 and ART12
Study duration: 10 days
Number of Groups: 4
Number of animals per group: 15
Total number of animals: 60

TABLE 6
Study Design.

| | | | | Group | |
|---|---|---|---|---|---|
| Group | Treatment | MPTP | Treatment | Size | Evaluations/Endpoints |
| 1 | Sham | PBS, 4 doses IP | Saline, | 15 | 3× per week: Body |
| | | QD, for 4 days | PO BID | | weight and |
| | | beginning day 0 | from | | clinical observations |
| 2 | MPTP | 4 doses, | Day (−2) | 15 | Day 6 post first MPTP dose |
| | | 20 mg/kg IP | to | | Clean catch fecal pellet |
| | | QD for4 days | Day 7 | | collection (n > 3) |
| 3 | | beginning | ART24, | | 7 days post-first dose of MPTP: |
| | | day 0 | PO BID | | N = 15/group |
| | | | from | | Blood/plasma collection |
| | | | Day (−2) | | Dissection of striata (bilateral), |
| | | | to Day 7 | | (RT and LT separate) |
| 4 | | | ART12, | | Determination of |
| | | | BID QD | | DA/DOPAC/HVA in the striata |
| | | | from | | Cecal content collection |
| | | | Day (−2) | | Colon collection (flushed, |
| | | | to Day 7 | | divided, in proximal and distal |
| | | | | | pieces, weighed and suap |
| | | | | | frozen) |
| | | | | | Small intestine collection |
| | | | | | (flushed, divided in duodenum |
| | | | | | and ileum regions weighed and |
| | | | | | snap frozen) |
| | | | | | Liver collected and snap frozen |

### Methods

MPTP model. MPTP was formulated in phosphate buffered saline (PBS) and administered IP to mice QD for 4 days at a dose of 20 mg/kg (2 mg/mL delivered as 10 mL/kg).

Tissue dissection. Mice from each group were anesthetized and blood collected by cardiac puncture. Blood was processed for plasma. Brains were dissected.

The brain was hemisected. The rostral part was dissected and striata removed (left and right side separately). The left and right striatum were weighted and frozen in separate tubes. Right striatum was subsequently evaluated for levels of dopamine and metabolites. Left striatum was collected in a separate tube and frozen. Left striatum samples were sent for total transcriptome analysis (RNAseq) Briefly, mRNA was isolated, cDNA libraries were constructed and sequenced, and the products were mapped to a murine reference genome. Gene expression quantification was performed and differential expression analysis was done. Enrichment analysis was performed to identify groups of genes related by pathway or disease state. Cecal content was collected in a separate tube and frozen.

Colon was flushed, divided in proximal and distal pieces, weighed and snap frozen. Proximal samples were sent for total transcriptome analysis (RNAseq) Briefly, mRNA was isolated, cDNA libraries were constructed and sequenced, and the products were mapped to a murine reference genome. Gene expression quantification was performed and differential expression analysis was done. Enrichment analysis was performed to identify groups of genes related by pathway or disease state. Small intestine was flushed, divided in duodenum and ileum regions, weighed, and snap frozen. Liver was collected and snap frozen.

On day 6 post first MPTP dose, clean catch fecal pellets were collected from all mice. For the collection, mice were placed on the sterile surface (sterile surgical pad) and feces (at least 3 pellets) were collected into the sterile tubes using sterile instruments.

Dopamine and Metabolites and HVA Analysis. Mice were anesthetized and blood collected into K2EDTA tubes by a cardiac puncture. Brains were extracted and dissected. The rostral part was dissected and striata removed. The left and right striata were frozen separately and right striata subsequently evaluated for levels of dopamine and metabolites. Straia were homogenized in 0.3M perchloric acid. The resulting supernatant was analyzed for levels of monoamines by reverse phase HPLC-ECD. Blood was processed for plasma. Resulting plasma was snap frozen for further analyses.

Data analysis. Data were analyzed using a one-way ANOVA with a Fisher's LSD post hoc test. The raw data were also analyzed for outliers using ROUT method (Q=1%). GraphPad Prism 9 was used for data analysis and presentation.

### Results

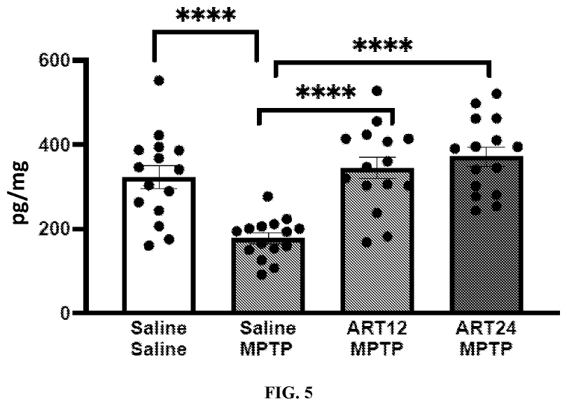

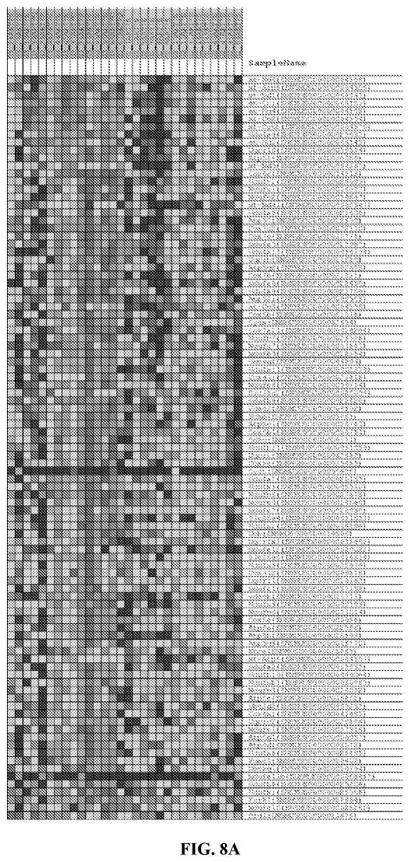

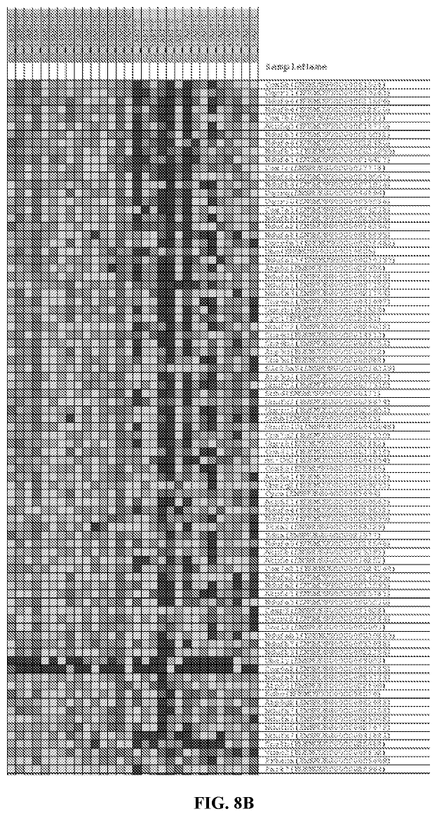

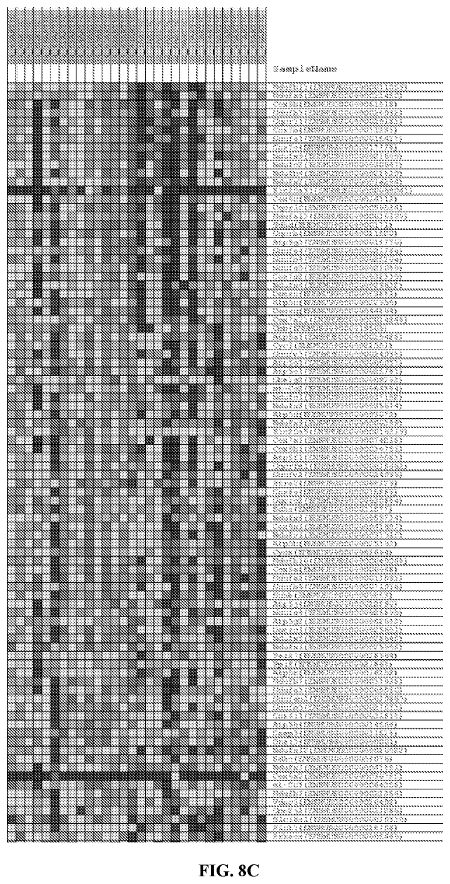

As shown in FIG. 5, both ART24 and ART12 significantly increase dopamine metabolite homovanillic acid (HVA) levels in the striatum of mice as compared to saline/MPTP dosed mice. As shown in FIGS. 8A-8C, treatment of MPTP dosed mice with ART12 increased Parkinson's Gene expression (KEGG grouping) in striatal tissue (ART12) and in colonic tissue (ART12 and ART24) indicating a stimulation of gene expression in pathways relevant to Parkinson's Disease.

Accordingly, these results demonstrate that ART24 and ART12 are effective in methods for preventing, reducing the risk of, or treating Parkinson's disease in a subject in need thereof.

### Example 8: Effect of ART24 on Dopaminergic Neurons in a Zebrafish Alpha-Synuclein Parkinson's Disease Model

The aim of this experiment was to evaluate rescue of the loss of dopaminergic neurons in the tuberculum area, induced by the overexpression of a mutated SNCA gene in zebrafish larvae treated with a pure lyophilized culture of ART24. This example demonstrates that ART24 is effective in methods for treating or preventing Parkinson's disease in an in vivo zebrafish model.

### Introduction

In this study, a transgenic zebrafish line, which overexpresses the alpha-synuclein gene (SNCA gene): Tg(L5cl3Nestin:GFP-2A-SNCAmut), as a model of Parkinson's disease (PD), was used. This line expresses human alpha-synuclein WT-EGFP in precursor cells of neurons through Nestin promoter. Alpha-synuclein (SNCA) is a neuronal protein that is thought to regulate snynaptic vesicle trafficking and subsequent neurotransmitter release. One of these neurotransmitters is dopamine, which is critical for controlling the start and stop of voluntary and involuntary movements. In humans suffering from PD, one of the most common mutations is a duplication of the alpha-synuclein gene, suggesting that alpha-synuclein can have a toxic gain of function effect.

### Materials and Methods

Materials. 60× E3 medium (for 2 L: 34.8 g NaCl, 1.6 g KCl, 5.8 g CaCl₂·2H₂O, 9.78 g MgCl₂·6H₂O, pH=7.2 (with NaOH 1M), 1xE3 medium: 16.5 mL of 60× E3 stock solution in 1 L of dH₂O); sodium citrate, SDS, goat serum, Phusion DNA polymerase (F-530S, ThermoFisher), PCR primers (IDT technologies), PBST, Tween 20 (Sigma), DIG-RNA labelling kit T7 (Roche), Bovine Serum Albumin (BSA), anti-digoxigenin-AP Fab fragments (Roche), NBT (Sigma), BCIP (Sigma), Methanol (Merck), Proteinase K, PFA 4% (Sigma), 20×SSC (Signma), Formamide (Sigma).

TABLE 7
PCR primers (IDT).

| Gene | Diagnostic PCR Primers |
|---|---|
| Th-Fw | AATCGGAACATGGCGGGAGG (SEQ ID NO: 1) |
| Th-Rv | TAATACGACTCACTATAGCAGTCCTGCTCCATACGCTTT |
| | (SEQ ID NO: 2) |

Test system. All experiments were conducted using zebrafish (Danio rerio) embryos as the experimental model.

Zebrafish maintenance. Adult Tg(L5cl3Nestin:GFP-2A-SCNAmut) zebrafish and their offspring were maintained at 28-29° C. on a cycle of 14 h light: 10 h dark (lights on at 7 am; lights off at 9 pm).

Treatment procedure. A minimum of 72 Tg(L5cl3Nestin:GFP-2A-SCNAmut) zebrafish larvae were treated from 72 hpf until 120 hpf, with medium replacement every 24 hours. At 120 hpf, larvae were fixed in 4% PFA to proceed with the in situ hybridization protocol (ISH).

Riboprobe synthesis and in situ hybridization (ISH). In vitro transcription of probes were performed using the RNA Labelling Kit (Roche) according to manufacturer's instructions. Dechorionated embryos at 120 hpf were fixed in 4% paraformaldehyde in 1×PBS for 4 hours at room temperature and whole mount in situ hybridization was carried out as described in Thisse & Thisse. Nat Protoc. 2008.

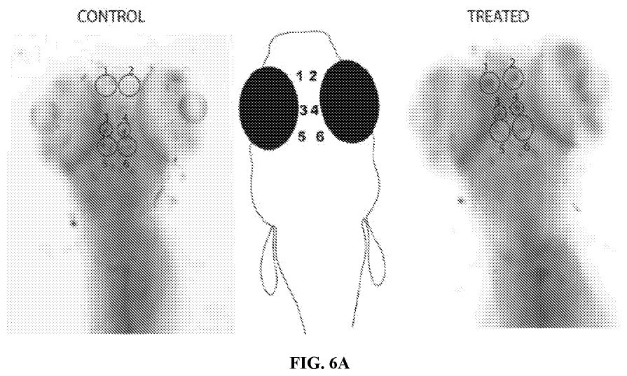

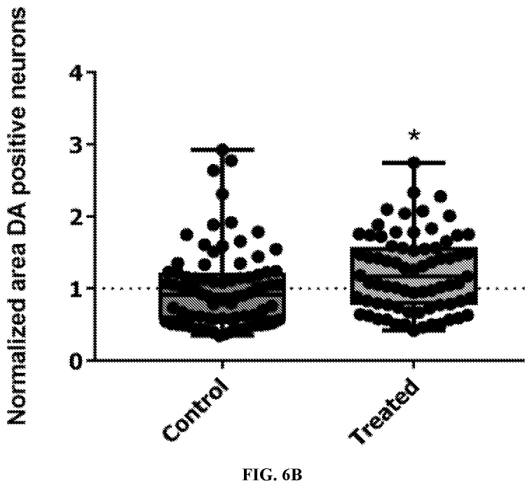

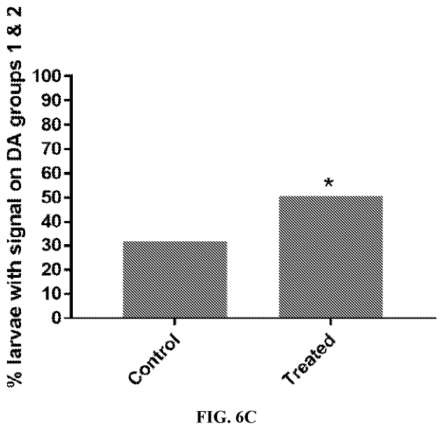

Imaging. After ISH, tyrosine hydroxylase (TH) expression patterns in whole larvae were imaged in a bright-field stereoscope. The images were analyzed with Fiji software in 6 different zones/clusters (FIG. 6A) for the expression of the ISH. In order to determine the overall expression pattern, the area of each zone was calculated. In each larva, all the areas from the 6 zones were summed and normalized against the average area of the control group. Larva having no TH expression in clusters 1 or 2 were annotated for further analysis.

Statistical analysis. Statistical analysis was performed using unpaired T-test (area of dopaminergic positive neurons) or Fisher's exact test (percentage of larvae with presence of neuronal clusters 1 and 2).

### Results

The Tg(L5cl3Nestin:GFP-2A-SNCAmut) zebrafish line was treated with the pure lyophilized culture of ART24 at 4×10⁶ CFU/mL in order to evaluate if it was producing a rescue on the loss of dopaminergic neurons in the tuberculum area. Thus, an in situ of the tyrosine hydroxylase was performed and its expression area in the tuberculum region was measured. Results show that Tg(L5cl3Nestin:GFP-2A-SNCAmut) zebrafish larvae treated with ART24 at 4×10⁶ CFU/ml had increased TH expression (FIG. 6A). In addition, the loss of the neuronal groups 1 and 2 (bilateral pretectal cluster), present in 30% of not treated Tg(L5cl3Nestin:GFP-2A-SNCAmut) zebrafish larvae, is also ameliorated by the treatment with ART24 (FIGS. 6B-6C). The loss of the bilateral pretectal cluster has also been shown in MPTP-treated zebrafish larvae (McKinley E T, et al., Brain Res Mol Brain Res. 2005).

### Conclusion

The results demonstrate that ART24 is capable of rescuing the loss of dopaminergic neurons in the tuberculum area induced by the overexpression of a mutated SNCA gene in zebrafish larvae. Accordingly, these results demonstrate that ART24 is effective in methods for preventing, reducing the risk of, or treating Parkinson's disease in a subject in need thereof.

### Example 9: Efficacy of ART24 and ART12 Strains on Movement in the MPTP Zebrafish Model of Parkinson's Disease

The aim of this experiment was to determine, by larval locomotion assessment, the efficacy of ART24 and ART12 in methods for treating or preventing Parkinson's disease in a zebrafish MPTP model.

### Materials and Methods

Materials. 60× E3 medium (for 2 L: 34.8 g NaCl, 1.6 g KCl, 5.8 g CaCl₂·2H₂O, 9.78 g MgCl₂·6H₂O, pH=7.2 (with NaOH 1M), 1xE3 medium: 16.5 mL of 60× E3 stock solution in 1 L of dH₂O); MPTP (Thermo scientific, 150687); 48 well plate; EthoVision XT12 software (Noldus Information Technologies, Wageningen, The Netherlands); DanioVision (Noldus Information Technologies, Wageningen, The Netherlands).

Test system. All experiments were conducted using zebrafish (Danio rerio) embryos as the experimental model.

Zebrafish maintenance. Adult wild-type zebrafish and their offspring were maintained at 28-29° C. on a cycle of 14 h light: 10 h dark (lights on at 7 am; lights off at 9 pm).

Behavioral study protocol. The zebrafish larvae locomotion was traced and analyzed by the EthoVision XT 12 software and the DanioVision device from Noldus Information Technologies, Wageningen, The Netherlands. This closed system consists of a camera placed above a chamber with circulating water and a temperature sensor that is set at 28° C. Larvae in a plate are placed in the chamber, which can provide different stimuli (light/dark environment, tapping, sound) controlled by the software.

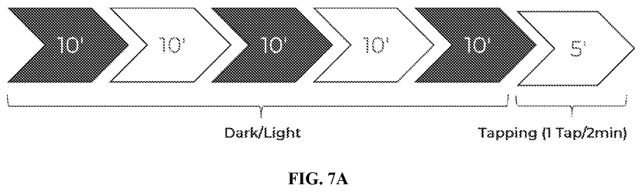

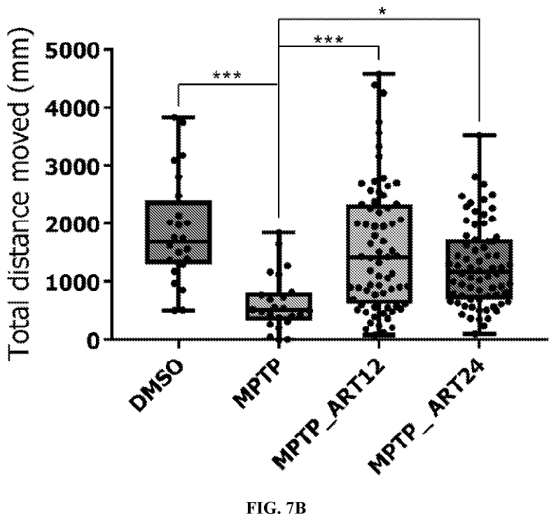

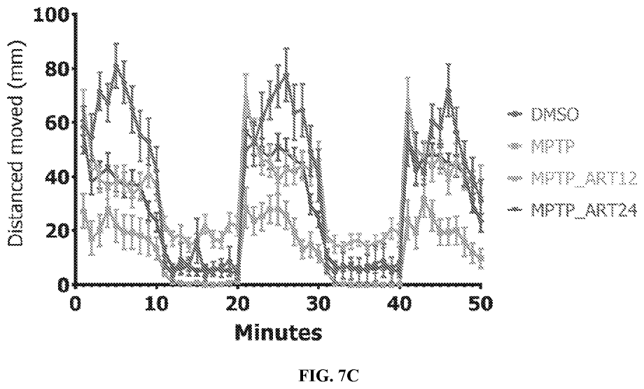

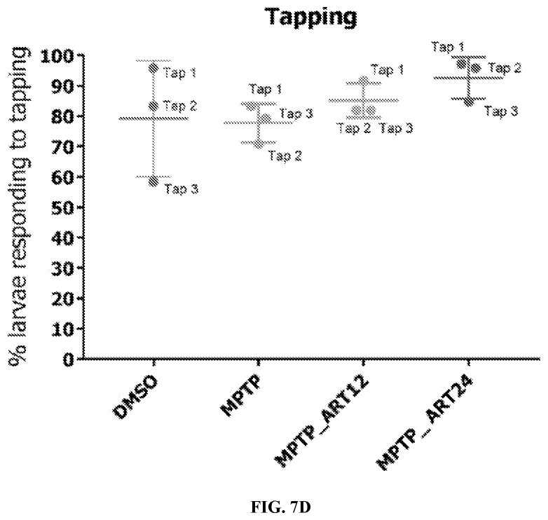

Twenty-four hours before the behavioral experiment individualized larvae in a 48-well micro plate were treated with DMSO (negative control), MPTP at 250 M (positive control), and MPTP at 250 M in combination with ART12 or ART24 at 4×10⁶ CFU/mL. Afterwards, a standard protocol of light/dark alternation was performed: 50 minutes (total time) divided in intervals of 10 minutes of altering light and dark stimuli (FIG. 7A). At the end of the experiment a tapping stimulus (three taps separated by 2 minutes) was added to measure startle response (FIG. 7A). It is a useful internal experimental control added to rule out the possibility that larvae in treatment conditions are not responding to the different light/dark stimuli due to severe motoneuron alterations.

Statistical analysis. Statistical analysis is performed using one-way ANOVA followed by Tukey's multiple comparison test.

### Results

Adult wild-type fish were crossed and descendants maintained until 4 days post fertilization (dpf). At 4 dpf, larvae were plated individualized in a 48-well plate and then treated. After 24 hours of treatment (5 dpf) larvae were placed into the DanioVision chamber where predetermined series of alternating dark and light environment were presented in order to evaluate their locomotion. Results are summarized in FIGS. 7B-7C.

### Conclusion

As shown in FIGS. 7B-7C, treatment with ART24 or ART12 rescues the locomotion in a zebrafish Parkinson's disease MPTP model to a level similar to the negative control (DMSO). Accordingly, these results demonstrate that ART24 and ART12 are effective in methods for preventing, reducing the risk of, or treating Parkinson's disease in a subject in need thereof.

### Biological Deposits

The Applicant requests that a sample of the deposited microorganisms should be made available only to an expert approved by the Applicant.

Bacillus amyloliquefaciens strain ART24 deposited with the National Collection of Industrial Food and Marine Bacteria (NCIMB Ltd.) (International Depositary Authority), Ferguson Building, Craibstone Estate, Bucksburn, Aberdeen, AB21 9YA, Scotland, on 21 Jun. 2018, under NCIMB Accession Number 43088.

Bacillus amyloliquefaciens strain ART12 deposited with the National Collection of Industrial Food and Marine Bacteria (NCIMB Ltd.) (International Depositary Authority), Ferguson Building, Craibstone Estate, Bucksburn, Aberdeen, AB21 9YA, Scotland, on 21 Jun. 2018, under NCIMB Accession Number 43087.

The deposits were made according to the Budapest treaty on the international recognition of the deposit of microorganisms for the purposes of patent procedure.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

Each and every publication and patent mentioned in the above specification is herein incorporated by reference in its entirety for all purposes. Various modifications and variations of the described methods and system of the present technology will be apparent to those skilled in the art without departing from the scope and spirit of the present technology. Although the present technology has been described in connection with specific embodiments, the present technology as claimed should not be unduly limited to such specific embodiments. Indeed, various modifications of the described modes for carrying out the present technology which are obvious to those skilled in the art and in fields related thereto are intended to be within the scope of the following claims.

What is claimed is:

1. A method for treating or preventing Parkinson's disease (PD) in a subject in need thereof, the method comprising administering to the subject a therapeutically effective amount of a pharmaceutical composition comprising bacterial strain ART24 (NCIMB Accession No. 43088), bacterial strain ART12 (NCIMB Accession No. 43087), or a combination thereof.

2. The method of claim 1, wherein the bacterial strain is lyophilized.

3. The method of claim 1 or claim 2, wherein the bacterial strain is in the form of a spore.

4. The method of claim 1 or claim 2, wherein the bacterial strain is in vegetative form.

5. The method of claim 1 or claim 2, wherein the bacterial strain is a mixture of the strain in the form of a spore and in the vegetative form.

6. The method of any one of claims 1-5, wherein the bacterial strain is formulated as a dietary supplement.

7. The method of any of claims 1-5, wherein the bacterial strain is formulated as an edible product.

8. The method of claim 7, wherein the edible product further comprises a carrier, vehicle, or excipient.

9. The method of claim 7, wherein the edible product further comprises a fermented food product, soybean, mushroom, mung bean, locus bean, rice, or extracts thereof.

10. The method of claim 9, wherein the soybean is fermented soybean or fermented soybean paste.

11. The method of claim 10, wherein the fermented soybean or fermented soybean paste is Cheonggukjang, Douchi, Hawaijar, Bekang, Peruyaan, Tungrymbai, Eoyukjang, Kinema, Aakhone, Miso, Natto, or Thua-nao.

12. The method of any one of claims 1-11, wherein the PD comprises one or more of tremors, muscle rigidity, slowed movement, poor posture and balance, changes in speech, struggle with fine motor skills, cognitive changes, constipation, early satiety, excessive sweating, fatigue, seborrheic dermatitis, hallucinations and delusions, orthostatic hypotension, loss of sense of smell or taste, depression, anxiety, apathy, irritability, insomnia, excessive daytime sleepiness, REM sleep behavior disorder, vivid dreams, and Restless Legs Syndrome, vision problems, and weight loss.

13. The method of any one of claims 1-12, wherein treatment or prevention comprises one or more of increasing striatal dopamine levels, increasing striatal DOPAC levels, or increasing striatal homovanillic acid levels.

14. The method of any one of claims 1-13, wherein the pharmaceutical composition is administered orally.

15. The method of any one of claims 1-14, further comprising separately, sequentially, or simultaneously administering one or more agents selected from the group consisting of a dopamine decarboxylase inhibitor, a dopamine precursor, a dopamine agonist, a cathechol-o-methyltransferase inhibitor, a monoamino oxidase-B inhibitor, an adenosine 2A antagonist, and anticholinergic, or an N-methyl-D-aspartate antagonist.

16. Use of a composition in the preparation of a medicament for treating Parkinson's Disease (PD) in a subject in need thereof, wherein the composition comprises bacterial strain ART24 (NCIMB Accession No. 43088), bacterial strain ART12 (NCIMB Accession No. 43087), or a combination thereof.

17. The use of claim 16, wherein the bacterial strain is lyophilized.

18. The use of claim 16 or claim 17, wherein the bacterial strain is in the form of a spore.

19. The use of claim 16 or claim 17, wherein the bacterial strain is in vegetative form.

20. The use of claim 16 or claim 17, wherein the bacterial strain is a mixture of the strain in the form of a spore and in the vegetative form.

21. The use of any one of claims 17-20, wherein the bacterial strain is formulated as a dietary supplement.

22. The use of any of claims 17-20, wherein the bacterial strain is formulated as an edible product.

23. The use of claim 22, wherein the edible product further comprises a carrier, vehicle, or excipient.

24. The use of claim 22, wherein the edible product further comprises a fermented food product, soybean, mushroom, mung bean, locus bean, rice, or extracts thereof.

25. The use of claim 24, wherein the soybean is fermented soybean or fermented soybean paste.

26. The use of claim 25, wherein the fermented soybean or fermented soybean paste is Cheonggukjang, Douchi, Hawaijar, Bekang, Peruyaan, Tungrymbai, Eoyukjang, Kinema, Aakhone, Miso, Natto, or Thua-nao.

27. The use of any one of claims 16-26, wherein the PD comprises one or more of tremors, muscle rigidity, slowed movement, poor posture and balance, changes in speech, struggle with fine motor skills, cognitive changes, constipation, early satiety, excessive sweating, fatigue, seborrheic dermatitis, hallucinations and delusions, orthostatic hypotension, loss of sense of smell or taste, depression, anxiety, apathy, irritability, insomnia, excessive daytime sleepiness, REM sleep behavior disorder, vivid dreams, and Restless Legs Syndrome, vision problems, and weight loss.

28. The use of any one of claims 16-27, wherein treatment or prevention comprises one or more of increasing striatal dopamine levels, increasing striatal DOPAC levels, or increasing striatal homovanillic acid levels.

29. The use of any one of claims 16-28, wherein the pharmaceutical composition is formulated for oral administration.

30. The use of any one of claims 16-29, further comprising separately, sequentially, or simultaneously administering one or more agents selected from the group consisting of a dopamine decarboxylase inhibitor, a dopamine precursor, a dopamine agonist, a cathechol-o-methyltransferase inhibitor, a monoamino oxidase-B inhibitor, an adenosine 2A antagonist, and anticholinergic, or an N-methyl-D-aspartate antagonist.

31. A composition used for treating Parkinson's Disease (PD) in a subject in need thereof, wherein the composition comprises bacterial strain ART24 (NCIMB Accession No. 43088), bacterial strain ART12 (NCIMB Accession No. 43087), or a combination thereof.

32. The composition for use of claim 31, wherein the bacterial strain is lyophilized.

33. The composition for use of claim 31 or claim 32, wherein the bacterial strain is in the form of a spore.

34. The composition for use of claim 31 or claim 32, wherein the bacterial strain is in vegetative form.

35. The composition for use of claim 31 or claim 32, wherein the bacterial strain is a mixture of the strain in the form of a spore and in the vegetative form.

36. The composition for use of any one of claims 32-35, wherein the bacterial strain is formulated as a dietary supplement.

37. The composition for use of any of claims 32-35, wherein the bacterial strain is formulated as an edible product.

38. The composition for use of claim 37, wherein the edible product further comprises a carrier, vehicle, or excipient.

39. The composition for use of claim 37, wherein the edible product further comprises a fermented food product, soybean, mushroom, mung bean, locus bean, rice, or extracts thereof.

40. The composition for use of claim 39, wherein the soybean is fermented soybean or fermented soybean paste.

41. The composition for use of claim 40, wherein the fermented soybean or fermented soybean paste is Cheonggukjang, Douchi, Hawaijar, Bekang, Peruyaan, Tungrymbai, Eoyukjang, Kinema, Aakhone, Miso, Natto, or Thua-nao.

42. The composition for use of any one of claims 31-41, wherein the PD comprises one or more of tremors, muscle rigidity, slowed movement, poor posture and balance, changes in speech, struggle with fine motor skills, cognitive changes, constipation, early satiety, excessive sweating, fatigue, seborrheic dermatitis, hallucinations and delusions, orthostatic hypotension, loss of sense of smell or taste, depression, anxiety, apathy, irritability, insomnia, excessive daytime sleepiness, REM sleep behavior disorder, vivid dreams, and Restless Legs Syndrome, vision problems, and weight loss.

43. The composition for use of any one of claims 31-42, wherein treatment or prevention comprises one or more of increasing striatal dopamine levels, increasing striatal DOPAC levels, or increasing striatal homovanillic acid levels.

44. The composition for use of any one of claims 31-43, wherein the pharmaceutical composition is formulated for oral administration.

45. The composition for use of any one of claims 31-44, further comprising separately, sequentially, or simultaneously administering one or more agents selected from the group consisting of a dopamine decarboxylase inhibitor, a dopamine precursor, a dopamine agonist, a cathechol-o-methyltransferase inhibitor, a monoamino oxidase-B inhibitor, an adenosine 2A antagonist, and anticholinergic, or an N-methyl-D-aspartate antagonist.

46. The method of any one of claims 1-15, the use of any one of claims 16-30, or the composition for use of any one of claims 31-45, wherein the bacterial strain is ART24.

47. The method of any one of claims 1-15, the use of any one of claims 16-30, or the composition for use of any one of claims 31-45, wherein the bacterial strain is ART12.
